(19) United States
(12) Patent Application Publication
(10) Pub. No.: US 2011/0237864 A1
(43) Pub. Date: Sep. 29, 2011

(54) Prosthetic Implant For Suburethral Support With Gussets

(75) Inventors: Laure-Cecile Martin, Ternay (FR); Jean-Paul Gilbert Ricol, Saint Germain Sur L'arbresle (FR)

(73) Assignee: Compagnie De Recherche en Composants, Implants er Materiels Pour L'Application Clinique, Vienne (FR)

(21) Appl. No.: 13/061,294

(22) PCT Filed: Sep. 1, 2009
(86) PCT No.: PCT/FR2009/051650

§ 371 Date: May 3, 2011

(30) Foreign Application Priority Data
Sep. 1, 2008 (FR) 0804788

(51) Int. Cl. A61F 2/02; B23P 11/00

(52) U.S. Cl. 600/30; 29/428

(57) ABSTRACT

The invention relates to a prosthetic implant (1) for suburethral support comprising a suburethral support element (3) of elongate form that extends longitudinally between a first end (3A) and a second end (3B), said first end (3A) at least having first and second opposite faces (4), said implant (1) being characterized in that said suburethral support element (3) comprises at least a first gusset (11), which projects from the side of said first face (4) of the first end (3A), and a second gusset (12), which projects from the side of said second face (5) of the first end (3A), said first and second gussets (11, 12) being designed to fasten said first end (3A) within biological tissues of the patient. Prosthetic implants.

### TECHNICAL FIELD

The present invention relates to the general technical field of devices that can be surgically implanted to support the urethra for the purpose of treating urinary incontinence, and notably for the purpose of treating feminine urinary stress incontinence.

The present invention relates more particularly to a suburethral supporting prosthetic implant comprising a suburethral supporting element of elongate shape extending longitudinally between a first and a second end, said first end at least having a first and a second opposite face.

The invention also relates to a method for manufacturing a suburethral supporting prosthetic implant comprising a step of manufacturing or of supplying a suburethral supporting element of elongate shape extending longitudinally between a first and a second end, said first end at least having a first and a second opposite face.

### PRIOR ART

Urinary incontinence corresponds to an involuntary loss of urine through the urethra. This involuntary loss is usually due to the fact that the tissues responsible for holding back the urine are no longer capable of exerting sufficient force to oppose the forces for expelling the urine. Urinary stress incontinence corresponds in particular to a loss of urine that is not preceded by a sensation of need and that does not occur at rest but during stress.

Amongst the surgical treatments proposed for correcting urinary incontinence, the treatment by urethral support with the aid of a tape is known in particular.

The tape in question is surgically inserted without tension under the middle portion of the urethra and is designed to support the latter during stress in order in this way to prevent any leakage of urine. There are several types of tapes associated with different surgical procedures. The TVT (Tension-free Vaginal Tape), associated with the surgical treatment by the retropubic procedure, and the TOT (Trans-Obturator Tape), associated with the surgical treatment by the trans-obturator procedure, are thus known, to cite only some of the most commonly used.

The method using the TVT tape is however relatively difficult to apply and has not inconsiderable operating risks because there is notably a risk of perforating the bladder when manipulating the needles used to insert the tape. That is why it is essential, with this surgical method, to carry out a cystoscopy. More serious consequences have even occasionally been reported, such as a perforation of the iliac artery or else of the small intestine, causing visceral complications that are sometimes fatal.

The surgical treatment using the TOT tape has a priori fewer risks of perforation of vital organs, but nevertheless does not completely eliminate the risk of vascular complications because of the length of the path occupied by the tape within the body.

In order to remedy the drawbacks of the TVT and TOT methods explained above, the applicant has recently proposed a minimally invasive method using a suburethral tape comprising a gusset at each of its ends.

This suburethral tape is relatively short and is designed to be inserted using the tension-free vaginal procedure, the tape being held in position inside the body of the patient simply by pressure and friction of the gussets on the biological tissues.

This tape generally gives satisfaction notably because it is of particularly simple and cheap construction while being effective and not very invasive.

Nevertheless it does have a certain number of drawbacks. Thus, in some cases and in certain conditions, a non-optimal hold of the tape within the biological tissues has been observed that might cause recurrences and/or vaginal erosions, the incorrectly held tape tending to rub against the vagina which causes erosions.

### SUMMARY OF THE INVENTION

The objects assigned to the invention consequently are to propose a novel suburethral supporting prosthetic implant that does not have the drawbacks mentioned above and that makes it possible to effectively correct urinary stress incontinence while reducing the risks of operating complications and of recurrence while being of particularly simple, reliable and cheap construction.

Another object of the invention is to propose a novel suburethral supporting prosthetic implant of which the design allows an excellent positional hold of said implant within the body of the patient.

Another object of the invention is to propose a novel suburethral supporting prosthetic implant of particularly simple construction.

Another object of the invention is to propose a novel suburethral supporting prosthetic implant that is particularly easy to insert while being extremely stable in position within the body of the patient once inserted.

Another object of the invention is to propose a novel suburethral supporting prosthetic implant that is particularly simple and cheap to manufacture.

Another object of the invention is to propose a novel suburethral supporting prosthetic implant that is capable of being put in place exclusively by vaginal procedure.

Another object of the invention is to propose a novel method for manufacturing a suburethral supporting prosthetic implant that makes it possible to obtain a suburethral supporting prosthetic implant that is particularly effective and is of simple and cheap construction.

Another object of the invention is to propose a novel method for manufacturing a suburethral supporting prosthetic implant the application of which, in particular in an industrial context, is particularly easy, quick and cheap.

The objects assigned to the invention are achieved with the aid of a suburethral supporting prosthetic implant comprising a suburethral supporting element of elongate shape extending longitudinally between a first and a second end, said first end at least having a first and a second opposite face, said implant being characterized in that said suburethral supporting element comprises at least a first gusset that protrudes from the side of said first face of the first end and a second gusset that protrudes from the side of said second face of the first end, said first and second gussets being designed to hook said first end into the biological tissues of the patient.

The objects assigned to the invention are also achieved with the aid of a method for manufacturing a suburethral supporting prosthetic implant comprising a step of manufacturing or of supplying a suburethral supporting element of elongate shape extending longitudinally between a first and a second end, said first end at least having a first and a second opposite face, said method being characterized in that it comprises a step of adjoining to said suburethral supporting element a first gusset that protrudes from the side of said first face of the first end and a second gusset that protrudes from the side of said second face of the first end, said first and second gussets being designed to hook said first end into the biological tissues of the patient.

### BRIEF DESCRIPTION OF THE DRAWINGS

Other advantages and objects of the invention will appear in greater detail on reading the following description and with the aid of the appended drawings supplied purely by way of illustration and not being limiting, in which:

FIG. 1 illustrates, in a view from above, a prosthetic implant according to a first embodiment of the invention in a state prior to being placed in its final form.

FIG. 2 illustrates, in a schematic view, a prosthetic implant according to the invention, which corresponds to the implant of FIG. 1 in its final form.

FIG. 3 illustrates, in a schematic view in longitudinal section, the prosthetic implant of FIG. 2.

FIG. 4 illustrates, in a view from above, an implant according to a second embodiment of the invention, in a state prior to being placed in its final form.

FIGS. 5 to 11 illustrate various steps of surgical insertion of the prosthetic implant of FIGS. 1 to 3.

FIG. 12 illustrates the prosthetic implant of FIGS. 1 to 3 accompanied by a surgical instrument intended to make it easier to insert.

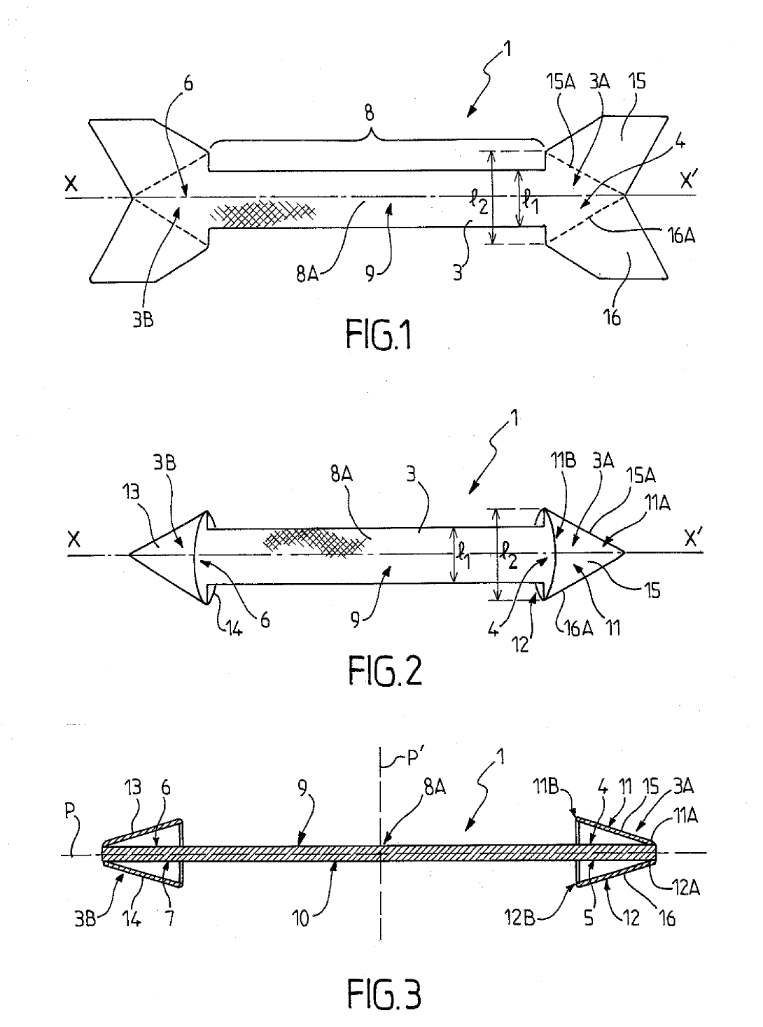

FIGS. 2 to 4 and 12 show a suburethral supporting prosthetic implant 1 according to the invention designed to be implanted into the body of a patient for the purpose of producing a urethral support making it possible to correct urinary incontinence and notably urinary stress incontinence in women.

### THE BEST WAY OF EMBODYING THE INVENTION

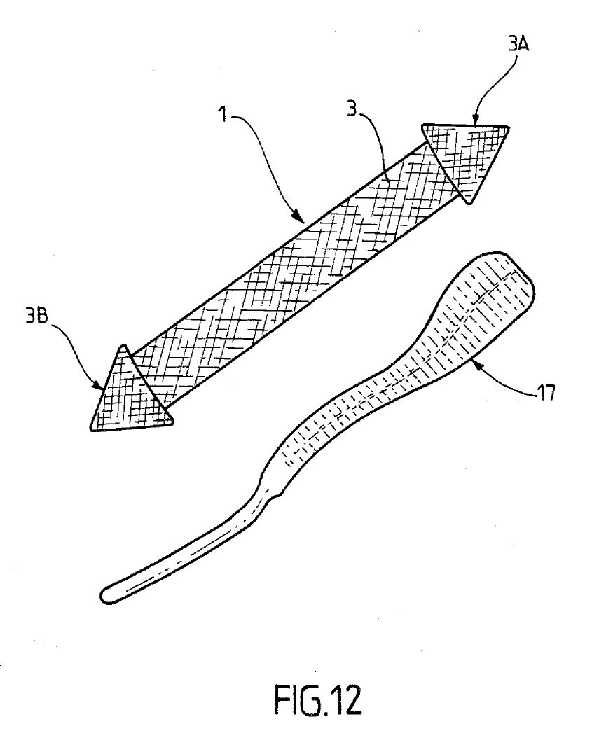

The term “prosthetic implant” in this instance designates a prefabricated prosthesis designed to be inserted into the body of the patient, preferably by surgical procedure. FIG. 11 illustrates the prosthetic implant 1 in its functional position, that is to say the position in which it will be used to support the urethra 2 and thus prevent involuntary urinary leakage. According to the invention, the suburethral supporting prosthetic implant 1 comprises a suburethral supporting element 3 of elongate shape extending longitudinally between a first and a second end 3A, 3B. Preferably, the suburethral supporting element 3 is formed by a textile tape as illustrated in FIG. 12. This tape is preferably made of knitted polypropylene and advantageously has an open structure, that is to say provided with openings delimited by various sets of threads. According to the invention, the suburethral supporting element 3 is substantially supple and more precisely is sufficiently supple and flexible to, on the one hand, prevent a phenomenon of shearing of the biological tissues after its insertion into the body, and, on the other hand, to allow it to follow the possible movements of the surrounding biological tissues after its implantation.

According to the invention, the first end 3A at least has a first and a second opposite face 4, 5. Advantageously, the second end 3B also has a first and a second opposite face 6, 7. The textile tape advantageously forming the suburethral supporting element 3 therefore preferably consists of a main tape 8 that is substantially rectangular and itself has a first and a second opposite face 9, 10. The first respective faces 4, 6 of the first and second ends 3A, 3B advantageously extend in line with the first face 9 of the main tape 8, in the same plane as the latter, while the second respective faces 5, 7 of the first and second ends 3A, 3B extend in line with the second face 10 of the main tape 8, in the same plane as said second face 10.

According to the invention, the suburethral supporting element 3 comprises at least a first gusset 11 that protrudes from the side of the first face 4 of the first end 3A and a second gusset 12 that protrudes from the side of the second face 5 of the first end 3A, said first and second gussets 11, 12 being designed to hook the first end 3A of the suburethral supporting element 3 into the biological tissues of the patient. In other words, the first end 3A extends in a mid-plane P and has faces 4, 5 that are situated on either side of said plane P, the first gusset 11 being situated on the side of the first face 4, while the second gusset 12 is situated on the other side of the plane P, on the side of the second face 5. In the variant illustrated in FIGS. 1 and 3, the first gusset 11 stands substantially at right angles to the face 4, so as to form a protuberance at the surface of the first end 3A on the side of its first face 4. Similarly, the second gusset 12 stands at right angles to the second face 5 so as to form a protuberance at the surface of the first end 3A, but this time on the side of its second face 5. It is not, however, necessary for the gussets to stand strictly at right angles to the faces 4, 5 of the first end 3A, the principle being that they are situated on either side of the extension mid-plane P in which the end 3A in question is substantially included. Preferably, the tape in its entirety extends along said plane P.

“Gusset” here means a small pocket which, in this instance, preferably opens on the side of the central portion 8A of the main tape 8.

The first and second gussets 11, 12 therefore form supple pockets which are deployed at the surface of said first end 3A in order to form a means for anchoring, by pressure and friction, said first end 3A in the biological tissues of the patient so as to hold the supporting element 3 in a determined position. The applicant has therefore found that the use of two gussets 11, 12 placed on either side of the first end 3A made it possible to obtain an excellent hold, reliable and comfortable for the patient, of said first end 3A of the supporting element 3. The gussets 11, 12 therefore make it possible on their own to hold the supporting element 3 in position by hooking itself to the surrounding biological tissues without it being necessary to make use of additional external means of the staple type. Moreover, the use of gussets 11, 12 as hooking means is likely to make the manufacture of the supporting element 3 easier as will be explained in greater detail below.

The use of gussets also makes the insertion easier because the gussets 11, 12 have the ability to flatten against the faces 4, 5 from which they respectively originate at the time of insertion which makes insertion easier. Conversely, the gussets 11, 12 also have the ability to deploy vertically from the faces 4, 5 from which they respectively originate, as illustrated in FIGS. 2 and 3 in order to fulfill an optimized hooking function in the tissues, preventing the unintended disengagement of the implant once the latter is inserted into the body of the patient.

Advantageously, the first and second gussets 11, 12 are substantially identical and are placed symmetrically relative to the first end 3A, that is to say that they are placed symmetrically relative to the mid-plane P in which the tape forming the suburethral supporting element 3 extends. In this case, the first and second gussets 11, 12 are advantageously situated at right angles to one another.

Preferably, each of said first and second gussets 11, 12 has a respective pointed front face 11A, 12A in order to make it easier to insert the first end 3A into the biological tissues of the patient.

Preferably, each of said first and second gussets 11, 12 also has a respective rear face 11B, 12B that is widened in order to form an anti-return abutment making it possible to hook the first end 3A into said biological tissues.

In other words, the first and second gussets 11, 12 are advantageously profiled in the form of a point toward the outside of the supporting element 3 so as to make it easier for said supporting element 3 to penetrate and move in the dissection space. Behind the point, each gusset 11, 12 has a widened base designed to rest against the biological tissues and thus prevent the textile tape forming the supporting element 3 from slipping. The first end 3A fitted with its first and second gussets 11, 12 therefore operates like an umbrella since it has an increased ability to enter the tissues in one direction (the insertion direction) while a certain resistance to moving back in the extraction direction, which ensures that the suburethral supporting element 3 is extremely well held in the body of the patient.

Advantageously, said suburethral supporting element 3 also comprises a third gusset 13 that protrudes from the first face 6 of the second end 3B and a fourth gusset 14 that protrudes from the second face 7 of the second end 3B, said third and fourth gussets 13, 14 being designed, like the first and second gussets 11, 12, to substantially hook said second end 3B of the suburethral supporting element 3 into the biological tissues of the patient.

Preferably, the third and fourth gussets 13, 14 are constructed in a symmetrical manner to the first and second gussets 11, 12.

This means that the suburethral supporting element 3 advantageously has not only a symmetry relative to the extension plane P but also a symmetry relative to the mid-plane P′ that is perpendicular to the plane P and that passes through the center 8A of the suburethral supporting element 3. The construction and the functions of the second end 3B and of the associated gussets 13, 14 are therefore identical to those of the first end 3A and of the associated gussets 11, 12, so that it does not seem necessary to describe the second end 3B and its gussets 13, 14 in greater detail because of the symmetrical character of the suburethral supporting element 3.

Advantageously, the first end 3A, and preferably the second end 3B, are, at least partly, wider than the rest of the suburethral supporting element 3. For example, as illustrated in FIGS. 1 to 3, the suburethral supporting element 3 can take the form of a central rectangular tape 8 of substantially constant width l₁ (measured in the extension mid-plane P), the first end 3A having at least one portion with a width l₂ that is greater than the width l₁ of the central tape 8, as illustrated in the figures. For example, the first end 3A advantageously takes the form of a flat triangular part the base of which, of length l₂, extends perpendicularly to the longitudinal axis X-X′ of extension of the supporting element 3, an apex of the triangle being placed on said longitudinal axis X-X′. In this case, the first end 3A takes the form of a point which makes it easier to insert the supporting element 3 into the tissues while procuring, in the extension mid-plane P, an anti-return effect obtained by virtue of the lateral crank that the end 3A forms relative to the central tape 8.

Therefore, in this preferred variant embodiment illustrated in FIGS. 1 to 3, the first end 3A is provided with anti-return means both in the extension mid-plane P (said anti-return means in this case being formed by the widening of the first end 3A relative to the rest of the supporting element 3) and in the plane P′ perpendicular to the plane P (said anti-return means being formed in this plane by the first and second gussets 11, 12).

Preferably, at least one of said first and second gussets 11, 12 and preferably both first and second gussets 11, 12 extend over the whole face 4, 5 of the first end 3A from which they originate. This means in particular, in the case of the variants illustrated in the figures, that the gussets 11, 12 also extend at right angles to the widened zones (of width l₂) of the first end 3A, so that said gussets 11, 12 thus reinforce the anti-return effect procured by said widening in the plane P, and adding thereto an additional hooking component in the plane P′.

Advantageously, said first and second gussets 11, 12 are made in one piece and of the same material as said first end 3A, just like said third and fourth gussets 13, 14 are advantageously made in one piece with and of the same material as said second end 3B. One of the main values of making use of the gussets as a hooking means actually lies in the possibility of easily producing gussets on each face of the supporting element from one part in a single piece.

In the variants illustrated in the figures, said first and second gussets 11, 12 are therefore advantageously made from the textile tape forming the suburethral supporting element 3. It is however entirely envisagable that each gusset 11, 12, 13, 14 is formed by a separate and independent part fitted and attached to the textile tape by any appropriate method (sewing, heat-sealing, bonding or other method).

Advantageously, as illustrated in FIG. 1, said textile tape comprises a first and a second flange 15, 16 placed at the first end 3A, and more precisely on either side of said end 3A, symmetrically relative to the longitudinal axis of extension X-X′ of the supporting element 3. The first flange 15 is advantageously folded over the first face 4 of the first end 3A in order to form the first gusset 11, while the second flange 16 is folded over the second face 15 of the second end 3A in order to form the second gusset 12. For example, the first flange 15 is folded along a fold line 15A (shown in dashed lines in FIG. 1) so as to be folded over the first face 4, said first flange 15 then advantageously being sewn (or heat sealed, or bonded) to the first face 4 in order to form the first gusset 11. Similarly, the second flange 16 is folded along a fold line 16A (shown in dashed lines in FIG. 1) so as to be folded over the second face 5, said second flange 16 advantageously being sewn onto the second face 5 in order to form the second gusset 12.

It is therefore possible, from a single textile part in a single piece (illustrated in FIG. 1), to produce in a particularly simple manner, with the aid of simple folding and sewing operations, a supporting element 3 according to the invention, that has an excellent hold inside the body of the patient.

According to the variant illustrated in FIGS. 1 to 3, the suburethral supporting element 3 therefore advantageously takes the form of a central rectangular tape 8 extended by two triangles respectively forming the first and second ends 3A, 3B, the base of each of said triangles extending perpendicularly to the central tape 8, while the other two sides of the triangle form the fold lines 15A, 16A from which the flanges 15, 16 extend laterally (relative to the axis X-X′).

The latter are folded along their respective fold lines 15A, 16A so as to follow over the whole length the other fold line 16A, 15A and be sewn thereto, preferably over the whole of their length.

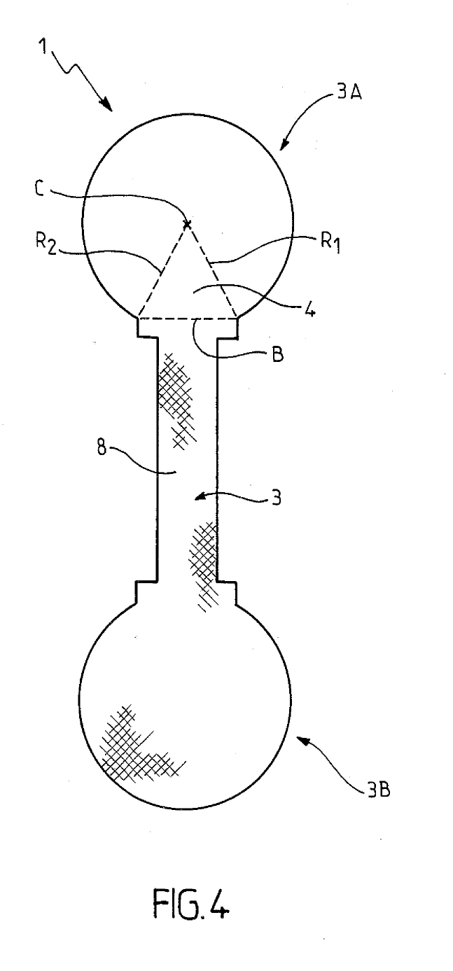

In the variant of FIG. 4, the textile tape takes the form of a part in a single piece consisting of a rectangular central tape 8 extended at each of its ends 3A, 3B by a substantially circular plate, with a center C with respect to the first end 3A. A cutout from said circular plate of center C is made on the line corresponding to the radius R₁. This gives a circular plate of center C provided with a notch over the whole length of its radius R₁. The circular plate thus split can then be made into the shape of a cone with a vertex C, said cone covering the triangle delimited by the dashed lines R₁, R₂ and B and being attached to the tape by the side R₂. The gussets 11, 12 are therefore in this case formed by a single cone that surrounds the faces 4, 5 of the end 3A and forms, on the side of the face 4, the first gusset 11, and, on the side of the face 5, the second gusset 12.

Advantageously, the overall length of the suburethral supporting element 3 is substantially between 90 and 140 mm, preferably of the order of 110 mm.

Advantageously, the average width of the suburethral supporting element is substantially between 9 and 20 mm, and preferably is of the order of 12 mm.

The prosthetic implant 1 according to the invention is advantageously inserted in the following manner.

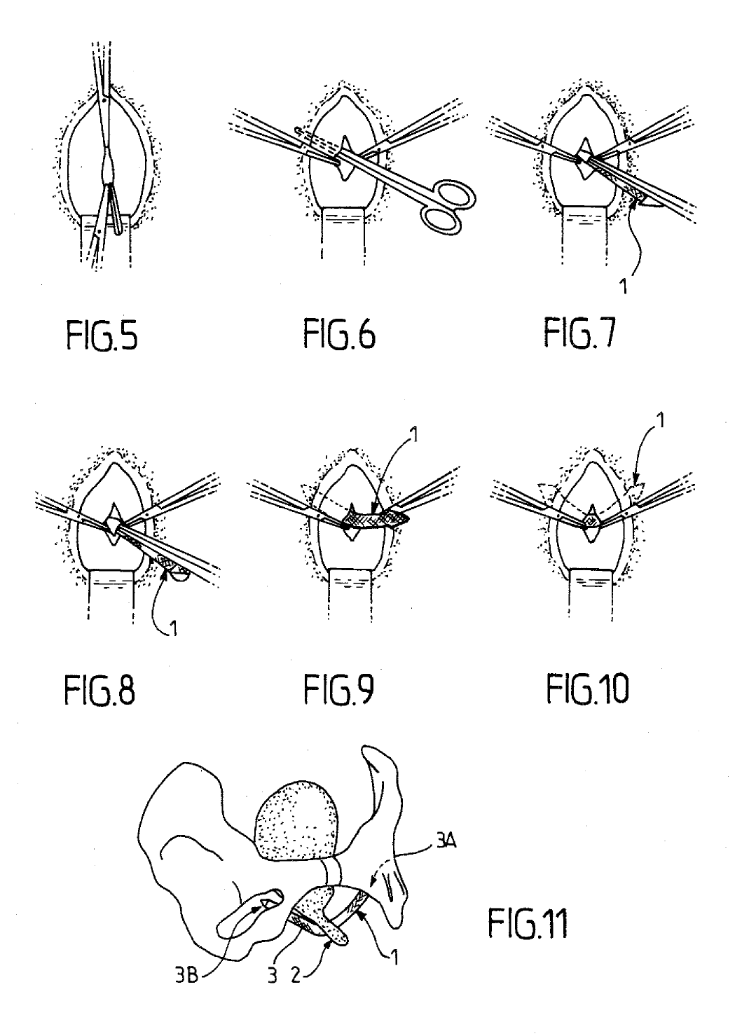

First of all, the patient is installed in a gynecological position, legs bent at 90 degrees. The description below of the operating actions taken relates to this position. A medium vertical incision approximately 2.5 cm deep is made on the internal vaginal wall at approximately 1.5 cm from the urinary meatus, as illustrated in FIG. 5. From this incision, the scissors are inserted laterally between the vaginal wall and the urethra, perpendicular to the ischiopubic branch, seeking contact with this boney branch. This step is illustrated in FIG. 6. This path is then pursued backward from this boney branch while keeping the same perpendicular axis and up to opening the fascia of pelvic muscles. There, the scissors are then opened carefully and then closed again before being withdrawn. The same actions are carried out on each side. As illustrated in FIG. 7, the point of the curved scissors is then inserted into one of the gussets at the end of the polypropylene tape forming the suburethral supporting prosthetic implant 1 according to the invention.

Thus sheathed, said tape is slipped into the path made in the preceding steps taking the end 3A of the tape up to the obturator muscles, through the fascia and without passing through the obturator membrane. The passage of the point of the scissors should be felt with a slight jump through the fascia. It ensures the correct positioning of each end 3A, 3B of the tape. As a variant, as illustrated in FIG. 8, it is possible to use an ancillary instrument 17 to relocate the dissection path, the ancillary instrument 17 being used as a guide for the scissors in order to bring the tape up to the obturator muscles, through the fascia and without passing through the obturator membrane. After this step, the ancillary instrument 17 is then withdrawn.

As illustrated in FIG. 9, the scissors are finally withdrawn, leaving the tape in the path. The same action is carried out in an identical manner on the other side. The tape is thus simply installed without tension, as illustrated in FIGS. 10 and 11. If the tension is insufficient, it is easy to reinsert the scissors at the bottom of the path into one of the gussets of the end in question and this will allow the appropriate repositioning.

The gussets 11, 12, 13, 14 provide an optimal anti-return effect which allows a stable positional hold of the tape inside the body of the patient.

This surgical method is minimally invasive, and therefore reduces the operating time and the associated risks of complications. These risks of complications are all the more reduced if the implant, once inserted, is at a distance from the vasculonervous package and the obturator membrane is not perforated. Postoperative neuralgic pain is thus substantially reduced.

Moreover, the quantity of implanted material is minimized because of the small dimensions of the tape compared with the tapes used in the previously known techniques.

The tape according to the invention is thus particularly suitable for the operating technique described above which is an exclusively vaginal insertion technique.

Finally, the invention also relates to a method for manufacturing a suburethral supporting prosthetic implant 1 and in particular a prosthetic implant 1 according to the invention.

The manufacturing method according to the invention comprises:
a step of manufacturing or of supplying a suburethral supporting element 3 of elongate shape extending longitudinally between a first and a second end 3A, 3B, said first end 3A at least having a first and a second opposite face 4, 5,
and a step of adjoining to said suburethral supporting element 3 a first gusset 11 that protrudes from the side of said first face 4 of the first end 3A and a second gusset 12 that protrudes from the side of said second face 5 of the first end 3A, said first and second gussets 11, 12 being designed to hook said first end 3A into the biological tissues of the patient.

Advantageously, said step of manufacturing or of supplying the suburethral supporting element 3 comprises the manufacture or the supply of a textile tape designed to form said suburethral supporting element 3, said tape comprising a first and a second flange 15, 16 placed at the first end 3A, said adjoining step comprising an operation of forming the gussets 11, 12 in which the first flange 15 is folded over the first face 4 of the first end 3A in order to form the first gusset 11, while the second flange 16 is folded over the second face 5 in order to form the second gusset 12.

The method according to the invention therefore makes it possible to manufacture in an extremely simple, rapid and easily industrializable manner a prosthesis having an excellent hold inside the body of the patient, based on a single textile part cut so as to adopt a shape conforming to that of FIG. 1, for example.

### INDUSTRIAL APPLICABILITY

The invention finds its industrial application in the design, the manufacture and the use of surgical implants.

1. A prosthetic implant for suburethral support comprising a suburethral supporting element of elongate shape extending longitudinally between a first and a second end, said first end at least having a first and a second opposite face, said implant being characterized in that said suburethral supporting element comprises at least a first gusset that protrudes from the side of said first face of the first end and a second gusset that protrudes from the side of said second face of the first end, said first and second gussets being designed to hook said first end into the biological tissues of the patient.

2. The implant as claimed in claim 1, characterized in that said second end has a first and a second opposite face, said suburethral supporting element comprising at least a third gusset that protrudes from the side of said first face of the second end and a fourth gusset that protrudes from the side of said second face (7) of the second end (3B), said third and fourth gussets being designed to substantially hook said second end into the biological tissues of the patient.

3. The implant as claimed in claim 1, characterized in that said first and second gussets are substantially identical and are placed symmetrically relatively to the first end.

4. The implant as claimed in claim 1, characterized in that each of said first and second gussets has a pointed front face in order to make it easier to insert the first end into the biological tissues of the patient and a rear face that is widened in order to form an anti-return abutment making it possible to hook said first end into said biological tissues.

5. The implant as claimed in claim 1, characterized in that said first and second ends are, at least partly, wider than the rest of the suburethral supporting element.

6. The implant as claimed in claim 1, characterized in that said first and second gussets are made in one piece with and of the same material as said first end.

7. The implant as claimed in claim 1, characterized in that the suburethral supporting element is formed by a textile tape and in that said first and second gussets are made from said textile tape.

8. The implant as claimed in claim 7, characterized in that the textile tape comprises a first and a second flange placed at the first end, the first flange being folded over the first face of the first end in order to form the first gusset, while the second flange is folded over the second face of the first end in order to form the second gusset.

9. The implant as claimed in claim 1, characterized in that the length of the suburethral supporting element is between 90 and 140 mm, preferably of the order of 110 mm.

10. The implant as claimed in claim 1 wherein the width of the suburethral supporting element is substantially between 9 and 20 mm, preferably of the order of 12 mm.

11. A method for manufacturing a suburethral supporting prosthetic implant comprising a step of manufacturing or of supplying a suburethral supporting element of elongate shape extending longitudinally between a first and a second end, said first end at least having a first and a second opposite face, said method being characterized in that it comprises a step of adjoining to said suburethral supporting element a first gusset that protrudes from the side of said first face of the first end and a second gusset that protrudes from the side of said second face of the first end, said first and second gussets being designed to hook said first end into the biological tissues of the patient.

12. The manufacturing method as claimed in claim 11, characterized in that said step of manufacturing or of supplying the suburethral supporting element comprises the manufacture or the supply of a textile tape designed to form said suburethral supporting element, said tape comprising a first and a second flange placed at the first end, said adjoining step comprising an operation of forming the gussets in which the first flange is folded over the first face of the first end in order to form the first gusset, while the second flange is folded over the second face in order to form the second gusset.

13. A prosthetic implant for use with a patient, the prosthetic implant comprising:
a suburethral supporting element having an elongate shape extending longitudinally between a first end and a second end, the first end at least having first and second opposing faces, said suburethral supporting element comprising:
at least a first gusset that protrudes from the side of the first face of the first end of said suburethral supporting element; and
a second gusset that protrudes from the side of the second face of the first end of said suburethral supporting element, said first and second gussets being designed to couple the first end of said suburethral supporting element into biological tissues of the patient.

14. The implant of claim 13 wherein the second end of said suburethral supporting element has first and second opposing faces and said suburethral supporting element comprises at least a third gusset that protrudes from the side of said first face of the second end of said suburethral supporting element and a fourth gusset that protrudes from the side of said second face of the second end of said suburethral supporting element, said third and fourth gussets configured to connect the second end of said suburethral supporting element into biological tissues of the patient.

15. The implant of claim 14 wherein said first and second gussets are substantially identical and are symmetrically disposed about the first end of said suburethral supporting element.

16. The implant of claim 13 wherein each of said first and second gussets has a pointed front face and a rear face that is widened in order to form an anti-return abutment making it possible to hook the first end of said suburethral supporting element into biological tissues of the patient.

17. The implant of claim 13 wherein at least portions of the first and second ends of said suburethral supporting element are wider than the rest of the suburethral supporting element.

18. A method for manufacturing a suburethral supporting prosthetic implant for use with a patient, the method comprising:
supplying a suburethral supporting element of elongate shape extending longitudinally between a first and a second end, the first end of the suburethral supporting element at least having first and second opposing faces;
adjoining a first gusset to a first one of the first and second opposing faces of said suburethral supporting element such that the first gusset protrudes from the first one of the first and second opposing faces of the first end of the suburethral supporting element, the first gusset having a shape configured to hook the first end of the suburethral supporting element into biological tissue of the patient; and
adjoining a second gusset to a second one of the first and second opposing faces of said suburethral supporting element such that the second gusset protrudes from the second one of the first and second opposing faces of the first end of the suburethral supporting element, the second gusset having a shape configured to hook the first end of the suburethral supporting element into biological tissue of the patient.

19. The method of claim 18 wherein supplying the suburethral supporting element comprises providing the suburethral supporting element from a textile tape, the textile tape comprising a first and a second flange placed at the first end.

20. The method of claim 19 wherein adjoining comprises:
folding the first flange over the first face of the first end of the suburethral supporting element to form the first gusset; and
folding the second flange over the second face of the first end of the suburethral supporting element to form the second gusset.
